(19) United States
(12) Patent Application Publication
(10) Pub. No.: US 2015/0104771 A1
(43) Pub. Date: Apr. 16, 2015

(54) SYSTEM AND METHOD FOR MONITORING AND TRAINING ATTENTION ALLOCATION

(71) Applicant: CARMEL-HAIFA UNIVERSITY ECONOMIC CORPORATION, LTD., Haifa (IL)

(72) Inventor: Amit Bernstein, Haifa (IL)

(21) Appl. No.: 14/518,498

(22) Filed: Oct. 20, 2014

Related U.S. Application Data
(63) Continuation of application No. PCT/IL2013/050342, filed on Apr. 18, 2013.
(60) Provisional application No. 61/636,121, filed on Apr. 20, 2012.

(51) Int. Cl. G09B 5/06; A61B 5/16; A61B 5/0484; G09B 19/00

(52) U.S. Cl. CPC G09B 5/06; G09B 19/00; A61B 5/165; A61B 5/0484
USPC 434/236

(57) ABSTRACT

A method and a system for monitoring and training attention allocation by applying at least one sensory stimulus over a human subject, using at least one stimulation device, where the sensory stimuli is associated with at least one attentional bias; measuring at least one attention allocation index of the subject by measuring response of the subject to the respective applied sensory stimulus; and outputting an attentional feedback indicative of the measured attention allocation indices. The feedback is outputted in real time or near real time, using one or more output devices for outputting the attention allocation feedback such as visual or auditory output devices.

### CROSS-REFERENCE TO RELATED PATENT APPLICATIONS

This application is a continuation from and claims priority to PCT Application No. PCT/IL2013/050342, filed Apr. 18, 2013, which claims priority from Provisional Patent Application Ser. No. 61/636,121 filed on Apr. 20, 2012, all of which is incorporated herein by reference in their entireties.

### FIELD OF THE INVENTION

The present invention generally relates to systems and methods for monitoring attention allocation of human subjects for improving awareness of subjects to their attention allocation.

### BACKGROUND OF THE INVENTION

Attentional Bias: Attentional bias has been conceptualized and operationalized as preferential allocation of attention to certain (target) stimuli, relative to competing (neutral) stimuli. Though bias is an adaptive capacity to preferentially allocate attention to important events (e.g. danger, appetitive cues), when deregulated, this mechanism drives psychopathology and addictions. The negative attentional bias literature has predominantly focused on visual spatial selective attention. The large majority of this literature has focused on attentional bias to supraliminal exogenous cues [˜200-1000 ms], such as threat and drug cues. In this context, spatial attentional bias results from facilitated engagement towards target stimuli, slower disengagement from target stimuli, and attentional avoidance away from target stimuli. From a functional perspective, it is noteworthy that problems with disengagement may most negatively impact behavioral functioning; and that a basic attentional dyscontrol mechanism contributes to problems with disengagement. Finally, research suggests that attentional processes underlying attentional bias involve both automatic and strategic processes, and thus may be controllable and modulated to some degree. Consistent with contemporary thinking regarding automaticity and control, these dual process systems are in fact necessarily inter-connected and inter-related. Thus, alleviating attentional dyscontrol may have strong effects on bias as well as on bias-mediated behavioral dysfunction (e.g., psychopathology, addictions).

Attentional Biases & Psychopathology: A well-established body of cross-sectional, longitudinal, experimental, and intervention research has demonstrated that biases of attention represent a core malleable bio-psycho-behavioral risk factor for multiple forms of psychopathology such as anxiety psychopathology and addictions, and related disorders. Greater levels of attentional bias are related to greater levels of psychopathology and various key psychopathogenic processes. Experimental reduction in bias causally results in lower levels of psychopathology and related psychopathogenic processes. This body of research thus provides evidence that attentional bias is a likely causal bio-psycho-behavioral risk factor for a variety of forms of psychopathology and addictions. Thus, attentional bias is one promising focus of the broader, field-wide search for core bio-psycho-behavioral processes underlying the etiology and maintenance of multiple prevalent and often co-occurring forms of psychopathology and addiction.

Attentional Bias Measurement—Behavioral Tasks: Some methodological means to identify attentional bias are well-developed and established. Four primary paradigms have been studied: (1) The visual probe detection task, also described as the dot-probe and visual probe task (see MacLeod, C., Mathews, A., & Tata, P. (1986). Attentional bias in the emotional disorders. Journal of Abnormal Psychology, 95, 15-20); (2) the emotion/addiction stroop (see: Hertel, P. T., & Mathews, A. (2011). Cognitive Bias Modification: Past Perspectives, Current Findings, and Future Applications. Persp. on Psych. Science, 6, 521-536)—both have been studied most extensively; (3) the emotional spatial cueing (see: Fox, E., Russo, R., Bowles, R., & Dutton, K. (2001). Do threatening stimuli draw or hold visual attention in subclinical anxiety? Journal of Experimental Psychology General, 130, 681-700); and (4) visual search tasks (see: Fox et al, 2001, and Wolfe, J. M. 1994. Guided Search 2.0: A revised model of visual search. Psychon. Bull. & Rev., 1, 202-238).

In the dot-probe task participants are instructed to focus on a centrally presented fixation cross, then stimuli (images, words) are briefly (500 ms) presented at the top and bottom (alternatively, right and left) of the monitor, after stimuli offset a probe appears in location of one of the two stimuli. Participants are asked to indicate the location of the probe by pressing one of two buttons (top vs. bottom/left vs. right). Preferential or biased allocation of attention is inferred from faster response times in congruent trials in which the probe replaces a target cue (e.g., threat), relative to incongruent trials in which the probe replaces the neutral (not target) cue.

In the (modified) spatial cueing task participants are instructed to focus on a fixation point located between two rectangles, a cue is then presented within one of the rectangles, followed by the brief (e.g., 500 ms) presentation of a target stimulus (e.g., threat cue, drug cue) within one of the rectangles. Participants are instructed to indicate in which of the two rectangles the target stimulus appeared. For “valid” trials, the pre-target cue draws attention to the rectangle in which the subsequent target stimulus will be located; whereas for “invalid” trials, the pre-target cue draws attention away from the rectangle in which the target will be located. Preferential or biased allocation of attention is inferred from faster responses (reduced latency) on valid threat/drug-cued trials relative to neutral-cued trials; as well as slower responses (increased latency) to invalid threat/drug-cued trials relative to neutral-cued trials.

Finally, the (modified) visual search task involves a variety of similar tasks that share the basic feature in which participants are asked to detect a target stimulus (words, images) spatially embedded within a matrix of distracting (e.g., neutral) stimuli; alternatively, a neutral target may be spatially embedded in a matrix of target (e.g., threat, drug) stimuli. This matrix, for example, may be a circle of distracters and target stimuli, or a matrix of rows and columns. Attentional bias is inferred from faster response times to detect a target stimulus in a matrix of neutral stimuli relative to detect a target neutral target stimulus in a neutral matrix; bias is also inferred from slower response times to detect a target neutral stimulus in a matrix of threatening stimuli relative to response times to detect a target neutral stimulus in a matrix of neutral stimuli.

Interventions Therapeutically Targeting Attentional Biases: In contrast to our knowledge of attentional biases and their role in psychopathology, the existing knowledge and technological means to systematically affect efficient, large, and lasting change in attentional biases for the purpose of reducing the development and maintenance of multiple forms of psychopathology are, highly limited. Limits of existing knowledge and technology similarly limit means to impact other important adaptive and maladaptive behaviors mediated by attentional bias beyond psychopathology (e.g., food-seeking appetitive behaviors, threat avoidance). Established therapeutic modalities targeting psychopathology, including psychotherapy and pharmacotherapy, have demonstrated mixed, and at best, small to modest therapeutic effects on attentional bias; moreover, these effects likely reflect the effect of reduced psychopathology on attentional bias rather than the effect of reduced attentional bias on psychopathology. This major limitation of extant psycho- and pharmaco-therapies led to the first significant innovation in the clinical science of attentional bias.

Attentional Bias Modification Training. Attentional Bias Modification Training (ABMT), a form of Cognitive Bias Modification Training (which furthermore entails biases in memory and interpretation), reflects the first major clinical science innovation to attempt to target attentional biases. ABMT is grounded in models of implicit conditioning. ABMT implicitly conditions a person's attention away from target stimuli (e.g., threat cues) and towards neutral or positive cues. It does so, for example, by using the dot-probe task, ABMT manipulates the location of the probe to appear exclusively in the spatial location of the neutral cue; this is in contrast to the standard use of the dot-probe task in which the probe randomly occurs in the locations of either the target or neutral stimulus. In short, ABMT conditions participants and patients to look away from target stimuli and towards neutral/competing stimuli on the specific task in which training is delivered. ABMT has been studied most frequently with respect to anxiety, and most commonly social anxiety and generalized anxiety disorders, and bias to threat cues as well as, but less so, with respect to addictive and mood disorders.

Though pioneering, ABMT represents only one very initial and limited means to target attentional bias. First, though ABMT conditions attention away from and towards other cues within a given attentional task, it does not build the capacity to monitor nor self-regulate biased attentional allocation—and thus it does not target the central mechanisms of attentional dyscontrol underlying bias. Second, we lack evidence regarding the generalization of bias reduction conditioned within a specific paradigm (e.g., dot-probe) to other paradigms (e.g., visual search) for which no implicit conditioning was delivered—calling into question whether the bias is extinguished beyond the conditioning paradigm. Third, there is very limited evidence of durable (over-time) bias reduction; notably, bias reinstatement effects are likely as implicit conditioning results in highly context-specific extinction of bias. Fourth, the magnitude or size of effects of ABMT on attentional bias are typically small. Thus the clinical significance of ABMT may ultimately prove to be relatively modest. Fifth, implicit conditioning attention away from a given target (e.g., threat) cue may in fact be contraindicated to achieve its long-term therapeutic aims. ABMT may incidentally promote a form of maladaptive attentional avoidance. Because attentional avoidance is thought to maintain anxiety psychopathology via limiting adaptive, elaborative processing of feared stimuli and its salutary processes (e.g., reappraisal), it may maintain or strengthen associations with threat or drug cues. Finally, ABMT scholars make clear that there is no theoretical argument, empirical evidence, nor neurobiological rationale that ABMT is the only means, necessarily the optimal, nor most effective means to reduce attentional bias.

US patent application No. 2011/0105937 by Pradeep et al, discloses a system that analyzes controlled and automatic attention for introducing stimulus materials (mainly media materials such as video, audio etc.). This system analyzes neuro-response measurements (such as electroencephalography (EEG)/event-related potential (ERP) based measurements) from subjects who are exposed to stimulation that elicit controlled and automatic attention. The purpose of this system is to identify location over the media that is associated with “high controlled attention metrics” and placing introduction stimulus materials in those locations. Another US patent application No. 2009/0327068 by Pradeep et al discusses a similar system that allows performing stimulus targeting using neuro-psychological and neuro-behavioral data taken from various available measuring systems and methods such as EEG/ERP, Galvanic Skin Response (GSR) etc.

There are patents and patent applications that discuss tracking of subjects' responses to various visual or other sensory stimuli for analyzing various psychological and/or other characteristics of the subjects, such as a patent application No. WO2007/040857 by Ghajar Jamshid. Ghajar discloses a system for testing cognitive impairments of subjects by providing the subject with multiple stimuli including a smoothly moving object, while tracking the subject's eye-movements. An analysis of the movements allows determining if the subject has a cognitive impairment.

Other patents and applications use stimuli-response analysis for improving image analysis and/or presentation such as US patent application No. 2011/206283, by Quarfordt et al, which teaches a method and a system for improving image analysis using gaze-data feedback; and/or U.S. Pat. No. 7,822,783, which discloses a method and apparatus for real time adaptation of presentation to individuals or US patent application no. 2009/0024049, disclosing combining a multiplicity of modalities of responses to stimuli of the nervous system.

US patent application No. 2008/0275358 by Freer et al teaches a training method for employing brainwave monitoring. According to Freer, a brainwave monitor is employed for determining level of attention and providing a training environment, in which the trainee is provided with a feedback indicating to the trainee whether he/she is in focus or not, while providing the trainee with an incentive to stay focused. In this case, the degree to which the subject is focused is measured and not the attention allocation of the trainee or degree to which their allocation is biased or preferential to certain cues or predefined stimuli.

Research by Rothermund (Rothermund, Klaus, “Motivation and Attention: Incongruent Effects of Feedback on the Processing of Valence”, Emotion, Vol 3 (3), September 2003, 223-238) tested the influence of feedback (inducing related motivational state of a subject) on evaluative decision. In this research, each subject was given positive/negative task-performances feedback (such as success or failure feedbacks) for inducing his/her motivational state in respect to each exercise/task for measuring his/her following performances in a valence related task. In Rothermund's experiments only task-performance feedback was given for the purpose of manipulating the motivation of the subject in order to check the subject's subsequent attention allocation to valence of stimuli such as a word that is either congruent or incongruent in valence with the induced motivational state.

Dibartolo (Dibartolo, Patricia Marten, “Effects of Anxiety on Attentional Allocation and Task Performance: an Information Processing analysis using Non-Anxious and Generalized Anxiety Disorder Subjects”, Dissertation Abstracts International: Section B: The Sciences and Engineering. August 1996, pp. 1435) studied attentional allocation and its influence on normal comparison (NC) subjects and subjects who suffer from generalized anxiety disorder (GAD). In particular the impact of neutral distractor and negative feedback cues on performance of an attention vigilance task was investigated. Individuals with GAD evidenced impaired performance on an attention vigilance task relative to NC subjects when neutral distractor cues were presented. Contrary to prediction, no group differences in performance were detected under conditions in which subjects were presented negative feedback cues they were told were relevant to their performance. Instead, GAD participants exhibited improvement during the experimental task such that their performance was equivalent to NC subjects.

A US patent application No. 2011/027765 by Nader Amir discloses systems for treating patients with an anxiety disorder that comprise a screen for displaying sets of stimuli, a computer to control the display of stimuli onto the screen during at least one treatment session and the ability for the patient to interact with the screen in response to the displayed stimuli. The interaction of the patient with the system during the treatment session is capable of treating patient anxiety associated with an anxiety disorder, such as social anxiety. The interaction includes, for example responding to other stimulations of interactivity appearing on the screen or interacting with a human therapist.

### SUMMARY OF THE INVENTION

According to one aspect of the present invention, there is provided a computerized method of monitoring and training attention allocation by applying at least one sensory stimulus over a human subject, using at least one stimulation device, where the sensory stimulus is associated with at least one attentional bias; measuring at least one attention allocation index of the subject by measuring response of the subject to the applied sensory stimulation, using at least one measuring device that measures physiological response of the subject to the respective stimulus, wherein data indicative of the measure response to the stimulus is outputted by the measuring device; deducing at least one attention allocation index measure from the measured physiological response to the stimulus, using at least one computer processor; and outputting an attentional feedback indicative of the deduced at least one attention allocation index of the subject, wherein the feedback is outputted in real time or near real time, using at least one output device.

Optionally, the sensory stimulus comprises at least one of: visual stimulation, auditory stimulation, tactile stimulation, olfactory stimulation, and/or gustatory stimulation.

Additionally or alternatively, the feedback is a visual feedback, auditory feedback, and/or tactile feedback outputted by using visual, auditory and/or tactile output devices, respectively.

According to some embodiments of the present invention, the attention allocation index comprises at least one of: (a) reaction time indicative of the time it took the subject to respond to the applied stimulus; (b) task time, indicative of the time it took the subject to fulfill a requested task associated with the respective applied stimulus; and/or (c) at least one physiological measure indicative of physical response of the subject to the stimulus.

Additionally or alternatively, the training is carried out by using a training program that comprises a set of training sessions each session includes a set of tasks comprising: (a) providing the subject with at least one sensorial stimulation; (b) measuring at least one attention allocation index by measuring the subject's response to the stimulation; and (c) outputting a feedback indicative of the measured at least one attention allocation index.

Optionally, the attention allocation index measuring is carried out by using at least one predefined attention allocation scheme, where the scheme is based on at least one of: a dot-probe paradigm; a spatial cueing paradigm; a visual search paradigm; and/or modified stroop task; interference based schemes, attentional inhibition based schemes.

According to some embodiments of the present invention, the measuring of the subject's attention allocation index(ices) is carried out by using an eye tracking system that measures the subject responses to visual stimuli.

Optionally, the response to a respective applied stimulus is measured by using at least one measuring device that measures physiological response of the subject to the respective stimulus, wherein data indicative of the measure response to the stimulus is outputted by the respective measuring device and is used to deduce the respective at least one attention allocation index measure therefrom.

The measuring device may comprise one of: an eye-tracking system, an MRI device, a psychophysiological device, or an EEG/ERP device and the like.

Optionally, the method further comprises recording values of measured attention allocation indices and calculating statistical values associated therewith.

According to another aspect of the present invention, there is provided a system for monitoring and training attention allocation that comprises: (a) at least one stimulation device capable of applying at least one sensory stimulus; (b) at least one computer processor enabling to operate a designated Attention Feedback Awareness and Control Training (AFACT) application, wherein the AFACT application enables monitoring at least one attention allocation index of a human subject by measuring response of the subject to applied sensory stimulus, wherein the sensory stimulus is associated with at least one attentional bias, and outputting an attention feedback indicative of said measured at least one attention allocation index of the subject in real time or near real time; and (c) at least one output device for allowing outputting the attention allocation feedback.

Optionally, the AFACT application applies stimuli through at least one output device connected thereto and performs said monitoring by measuring or receiving measured response time to the stimulus.

According to some embodiments of the present invention, the system further comprises at least one measuring device that measures physiological response of the subject to the respective stimulus, wherein data indicative of the measured response to the stimulus is outputted by the respective measuring device and is used to deduce the respective at least one attention allocation index therefrom. The measuring device may comprise at least one of: and eye-tracking system, an MRI device, a psychophysiological device, or an EEG/ERP device.

Optionally, the system further comprises a database storage comprising a multiplicity of training programs each adapted to a different attentional bias type. E such program may optionally be designed according to at least one additional input parameter inputted through predefined input fields provided by the AFACT application via a designated graphical user interface, wherein once these parameters are inputted, the AFACT application automatically selects and executes a suitable training program from the database according to the input parameters.

The AFACT may be operable through at least one remote server via at least one communication link, allowing thereby a multiplicity of end users to operate the application, according to some embodiments of the present invention.

According to yet another aspect of the present invention, there is provided a system for monitoring and training attention allocation of at least one subject that comprises at least one computer processor operating a monitoring and training application. The application comprises: (a) a monitoring module, which enables monitoring attention allocation of a human subject by measuring response of the subject to sensory stimuli in real time, where the sensory stimulus is associated with at least one attentional bias; and (b) a feedback module which receives the measured attention allocation in real time and enables outputting an attention feedback indicative of the measured attention allocation of the subject in real time or near real time.

Optionally, the monitoring module further enables applying the stimulus by controlling at least one stimulation device.

According to yet other aspects of the invention, there is provided a system for monitoring and training attention allocation of a subject including: a) at least one stimulation device capable of applying at least one sensory stimuli, wherein the stimulation device comprises at least one of: a computer screen, an audio speaker; b) at least one computer processor enabling to operate a designated Attention Feedback Awareness and Control Training (AFACT) application, wherein the AFACT application enables monitoring at least one attention allocation index of a human subject by measuring response of the subject to applied sensory stimuli, the sensory stimuli is associated with at least one attentional bias, and outputting an attention feedback indicative of the measured at least one attention allocation index of the subject in real time or near real time; and c) at least one output device for allowing outputting the feedback, wherein the output device comprises at least one of: a computer screen and/or an audio speaker.

### BRIEF DESCRIPTION OF THE DRAWINGS

FIG. 1 is a flowchart, schematically illustrating a method for monitoring attention allocation of a subject and providing real time feedback indicative of the monitored data, according to some embodiments of the present invention.

FIG. 2 is a flowchart schematically illustrating a process of real time/near real time monitoring and feedback presentation of attention allocation of a subject, using the dot-probe paradigm for monitoring the subject's attention allocation, according to one embodiment of the present invention.

FIGS. 3A-3D show four successive screenshots representing a process of a single task for monitoring attention allocation by using dot-probe based visual stimuli and monitoring, according to one embodiment of the present invention: FIG. 3A shows a gaze neutralizing screenshot; FIG. 3B shows a screenshot in which a target and neutral images are presented, according to a dot-probe based monitoring and stimulating technique; FIG. 3C shows a screenshot in which a probe is shown replacing the target image of the screenshot of FIG. 3B; and FIG. 3D shows a screenshot in which a feedback of the subject's performances in the respective task are visually represented in real time/near real time.

FIG. 4 shows some of the steps of the process of FIG. 2 showing how the feedback can be presented through a scale indicator showing “bias level” of the subject in real time/near real time, according to one embodiment of the present invention.

FIG. 5 schematically illustrates a system for monitoring attention allocation, according to some embodiments of the present invention.

FIG. 6 schematically illustrates a system for monitoring attention allocation, according to additional or alternative embodiments of the present invention.

FIG. 7 shows experimental results for measuring the attention allocation bias level of subjects who were provided with attention allocation feedback relative to subjects who were not provided with such feedback, according to one embodiment of the present invention.

### DETAILED DESCRIPTION OF THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

The present invention, in some embodiments thereof, provides methods and systems for monitoring and training attention allocation of human subjects and for providing real time feedback to each subject indicative of the monitored attention allocation, for improving his/her awareness to his/her attention allocation related processes and behavior. These novel methods and systems may be used for monitoring attention allocation and providing attention feedback that is associated therewith, for allowing subjects to practice their awareness to their attention allocation associated with the specific attentional bias, ultimately for improving their control over their response to stimuli associated with cues that are related to the specific attentional bias.

Increasing awareness to biased attentional allocation may improve subjects' capacity to self-monitor their attentional allocation and other behavioral aspects associated therewith, which can lead to increasing subjects' regulation and control of their biased attentional allocation. Improving control over one's responses to cues of stimuli associated with specific attentional biases may be a powerful training tool for helping subjects to neutralize psychopathogenic effects of attentional bias on the development and maintenance of multiple forms of psychopathology and addictions or even prevent such psychopathologies from occurring. Training attention allocation by using real time/near real time feedback may have other therapeutic benefits such as promotion of attention mediated adaptive behaviors such as safety-related behaviors including, for instance, security-related behaviors (e.g. improving attention allocation to threat related cues), driving, flight, combat behaviors and the like, and/or improving mediated adaptive behaviors such as weight loss or drug seeking/quitting related behaviors.

Improving awareness to cues of stimuli associated with specific attentional biases and controlling response thereto may be used as a tool for intentionally conditioning a subject's mind to reduce, acquire, or strengthen attentional biases for improving their instinctive responses to specific cues associated with those specific biases. This can be used, for example, for training law enforcement professionals to improve their attentional allocation to cues related to selected biases such as potential threat, for decreasing their response time to threat related cues/stimuli, and the like. Other similar implications involve various behaviors that are mediated by biases in or preferential of attentional allocation such as a wide range of appetitive behaviors (e.g., food-seeking), driving, flight, or security/combat.

According to some embodiments of the present invention, the monitoring and training includes a set of tasks. In each task one or more stimuli are applied on or presented to the subject such as visual, auditory, olfactory and/or tactile stimulus, through one or more stimulation devices such as a screen, a speaker, a light emitting source, a vibrating devices for tactile stimulation, and the like. Parameters relating to the subject's response to the stimuli are measured and recorded (e.g. by measuring the time it took the subject to divert his/her gaze from a target image to a reference point or the subject's input response to a provided task). At least some of the stimuli applied on the subject is associated with attentional biases that are related to one or more predefined areas such as threat related cues (e.g. image, word or sound that are related to threat) or drug cues.

The attention allocation monitoring may include any one of the aforementioned paradigms such as the dot-probe paradigm, the emotional spatial cueing paradigm or the visual search based paradigm. Any type of known in the art technique, system or device may be used to apply the stimuli and/or to acquire attentional allocation monitoring measurements such as eye-tracking systems, sound systems, systems that can stimulate and/or measure biological responses of the subject such as psychophysiological (e.g., skin conductance), electroencephalography (EEG/ERP), magnetic resonance imaging (MRI) based systems and the like.

The feedback may be indicative of any one or more of the measured response related parameters and/or a calculation that includes any one or more of the deduced parameters. The feedback may be visual, auditory, and/or tactile, for example, reflecting one or more values of one or more attention allocation indices on recent trial(s) or task.

The systems and methods of the present invention can be designed especially to address one or more predefined areas or fields that cause biased attention allocation (defined hereinafter as “bias fields”) such as threat, addictions, or any other field that is often associated with attentional biases or preferential allocation of attention in the general population, specific subpopulation(s), or in unique individuals.

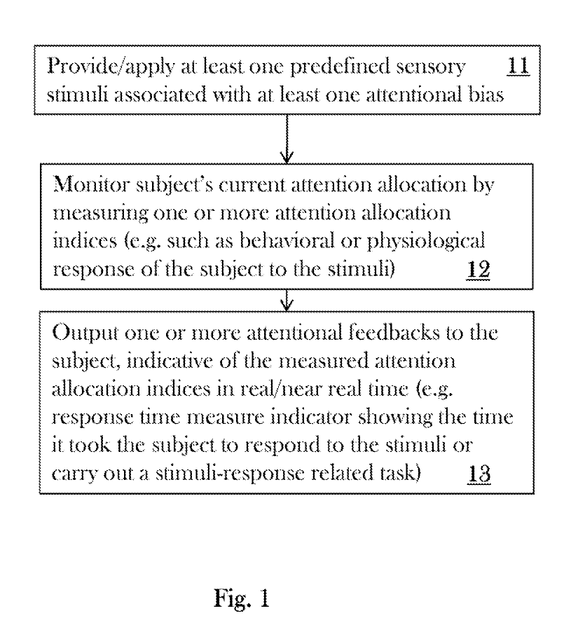

Reference is now made to FIG. 1, which is a flowchart, schematically illustrating a computerized method for monitoring a subject's attention allocation related to a specific predefined attentional bias, according to some embodiments of the present invention, using one or more processors (such as a computer system including a computer screen, input devices such as a keypad and/or a computer mouse or some response device and a processor and optionally auditory means such as a speaker—as known in the art). The method includes: (i) providing sensory stimulus associated with one or more specific and predefined attentional bias 11 (e.g. by using visual and/or auditory predefined cues associated with these attentional bias(es)); (ii) monitoring attention allocation of the subject in response to the provided stimulus by monitoring one or more attention allocation indices 12 such as, for example the subject's behavioral and/or physical response to the stimulus (e.g. by detecting responses such as eye movements, brainwaves, and the like or by receiving response input by the subject); and (iii) outputting (e.g. presenting) feedback indicative of the measured attention allocation such as the values of one or more attention allocation indices in real time or near real time to the subject 13.

The term “real time” or “near real time” means substantially immediately after the stimulus is applied. This means that at least some of the feedback associated with the respective stimulus will be provided to the subject before the next stimulus is applied. Therefore the feedback associated with the specific stimulus is given shortly after the measuring of the allocation of attention related parameters associated with the provided stimulus (e.g. the attention allocation measure to the specific stimulus such as the reaction time thereto or any other measurable parameter and/or accumulated measure including the measure of attention allocation associated with the specific stimulus).

The term “attention allocation index” refers to an observable indicator of attention allocation that can be measured using one or more devices and/or techniques such as (i) response time indicative of the subject's physiological or behavioral time of response to the applied stimulus (i); (ii) task time; (iii) task score such as the ability of the subject to correctly answer related questions immediately after being stimulated; (iv) physical measures of the subject such as brain activity, pulse and the like indicative of the subject's physical response to the stimulus; and/or any other measurable physiological and/or behavioral measures (parameters) indicative of the subject's attention allocation.

This process may be carried out using special software and/or hardware modules operated through one or more computers that allow outputting visual and/or auditory stimuli (such as presenting words or images over the computer screen of target and neutral cues as in the dot-probe paradigm) through devices of the computer such as the screen and/or speaker thereof. Once the target and neutral stimuli are simultaneously presented, the module either requires the subject to actively respond to the stimuli by requiring him/her to point the location of one of the stimulus cues presented (the neutral or the target cue) or automatically measures the response time by using response measuring equipment such as an eye-tracking system, a magnetic resonance imaging (MRI) based system and the like. The response parameter(s) (such as the response time) may then be presented to the subject in real time to provide the subject with an immediate feedback on his/her attention allocation with respect to the specific stimulus and response time.

According to some embodiments of the invention, as mentioned above, when using visual stimuli, e.g. in any one of the above-mentioned paradigms for measuring attention allocation of the subject in relation to a specific bias field, eye-tracking can be used to track the subject's attention allocation by tracking his/her eye movements. Eye-tracking of eye-movement can use simultaneous tracking of both the center of the pupil location and the corneal reflection, which together allow computation of gaze direction parameter. Temporally eye movements can be broken down into movements of the fovea on the visual field (saccades) and periods of relative stability during which an object can be viewed (fixations). Eye-tracking may provide a more direct indicator of overt visual attention and therefore of attention allocation relative to behavioral reaction-time. Moreover, eye-tracking permits measurement of attention allocation in a temporally continuous manner—measuring components (engagement, disengagement, avoidance) of attentional allocation in real-time, rather than via more temporally distal and gross behavioral indices (such as the user's active pressing over a key indicating the location of the probe as in the dot-probe monitoring paradigm).

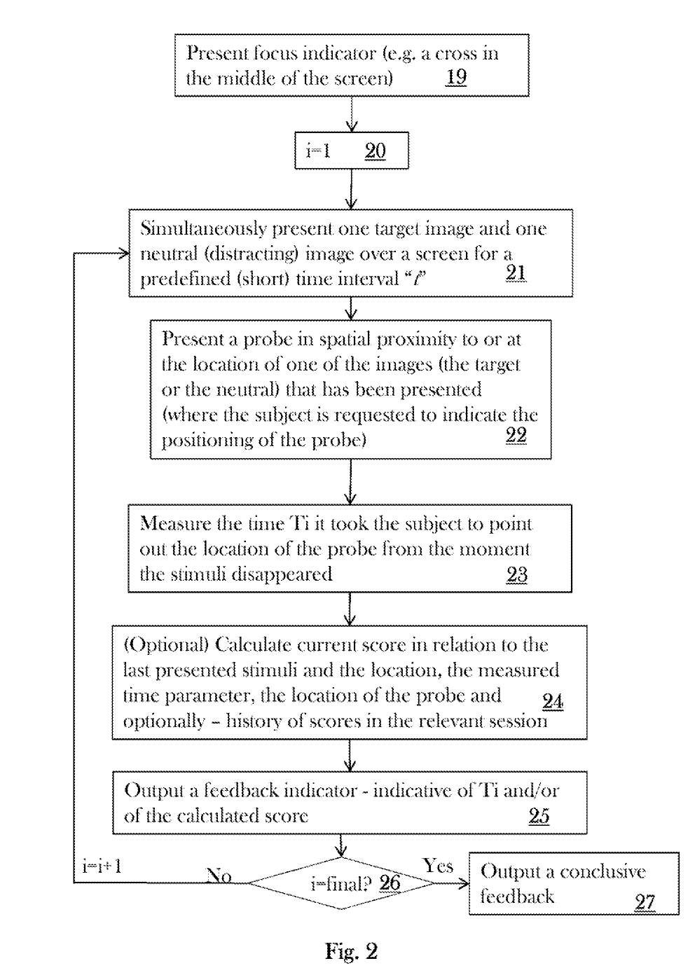

Reference is now made to FIG. 2, which is a flowchart schematically illustrating a process of real time/near real time monitoring and feedback presentation of (biased) attention allocation of a subject, using the dot-probe paradigm for monitoring the subject's attention allocation, according to one embodiment of the present invention. The process includes a set of a predefined number of tasks exercises each allows measuring response time of the user to a visual stimulus. Once the process is initiated 20, at each respective task “i”, two images are simultaneously presented to the subject 21 (e.g. over a computer screen) for a predefined short time interval “t”: a target image and a neutral (distracting) image e.g. representing target and neutral cue pictures/words respectively. A probe (or any other type of visual indicator) is then presented over the screen 22 at the location or in proximity to the location of one of the images (the target or the neutral) depending on the task definition (performing congruent or incongruent trials as described above). The subject is either requested as part of the session instructions to point the location of the probe or the identification of the probe location is carried out automatically (e.g. by using eye-tracking systems and techniques). The response time T1 is then monitored 23 by measuring the time it took the subject to identify and input the location of the probe. The response time T1 may be immediately presented over the screen for providing the subject with a real time feedback of his/her attention allocation performance at the specific task 25.

Additionally or alternatively, once each response time T1 is measured 23, a response parameter may be calculated 24 and presented 25 indicative of the subject's personal statistical response behavior (reaction-time, eye-tracking, brain activity or any other type of psychophysiological activity) taken by recording the values of the Ti index over time and calculating a statistical value representing the statistical attention allocation respective index.

Since some of the tasks in the same session of the dot-probe paradigm may be congruent with the target image location and some may be incongruent therewith, two averages may be calculated for each task type, since for subjects with proven attentional biases the response time in congruent tasks may be significantly shorter than the response time in incongruent tasks.

These separate mean parameters may also be indicative of the subject's attentional bias tendencies serving as a diagnostic tool for diagnosing the subject's bias level in addition to being a training tool by providing the subject with feedback indicative of these response related parameters.

Pointing out the location of the probe 22 (which is one of two location choices in the dot-probe based training such as up/down or left/right etc.) may be carried out by using a specific predefined marking/indicating technique such as using the computer mouse to bring the cursor to the location of the probe; using automatic eye-tracking technique; pressing one predefined key to indicate that the probe was in one optional location and another key for indicating that the probe was at the other optional location; using audio input (saying the position of the probe); and the like. The system then has the ability to receive the input and calculate the response time in relation to the trial type (congruent or incongruent) for deducing a corresponding attention measure allocation therefrom.

Optionally, as illustrated in FIG. 2, a neutralizing stimulus may be provided 19 at the beginning of the entire process (also referred to as session) or before each task “i”. The neutralization stimulus may include presenting a cross at the middle of the screen for neutralizing the subject's gaze before each task or before each session.

The time interval “t” may be constant or vary (e.g. decrease or increase) according to a predefined setup or according to the resulting measurements of the response time “T”.

Once all tasks have been executed 26, a “session performances feedback” may be presented to the user 27, indicative of the subject's attention allocation performances/behavior throughout the entire session. This parameter may include the average response time(s) of all tasks, for instance.

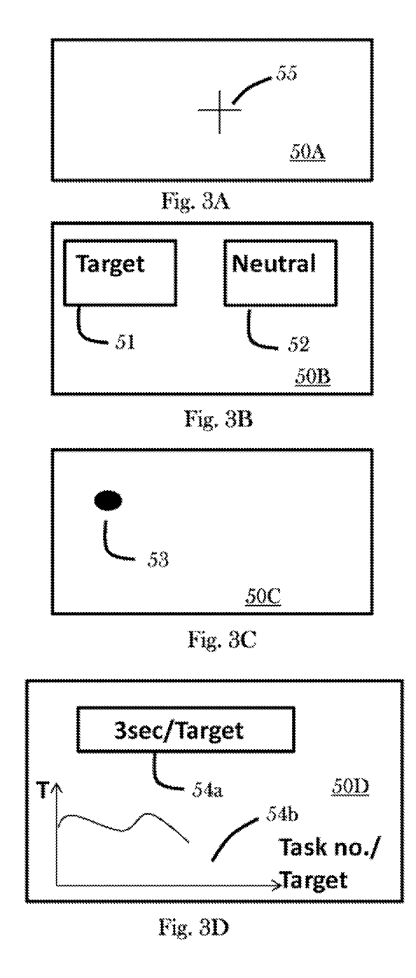

FIGS. 3A-3D visually illustrate the process of FIG. 2 by showing how some of the screens are represented throughout each task: FIG. 3A shows the gaze neutralizing screenshot 50A in which a cross 55 is presented at the center of the screen to neutralize/focus the subject's gaze. FIG. 3B shows another screenshot 50B in which the target and neutral images 51 and 52 respectively, are presented. FIG. 3C shows a screenshot 50C in which a probe 53 is shown replacing the target image 51 (to illustrate a congruent task type). FIG. 3d shows an optional screenshot 50D in which feedback parameters are presented to the subject. In this example, two parameters are shown: the current response time feedback parameter Ti 54a, indicative of the response time to the last task presented to the subject; and an accumulated response (attention allocation) performances feedback represented as a graph or a histogram 54b showing the measured response time vs. the task number.

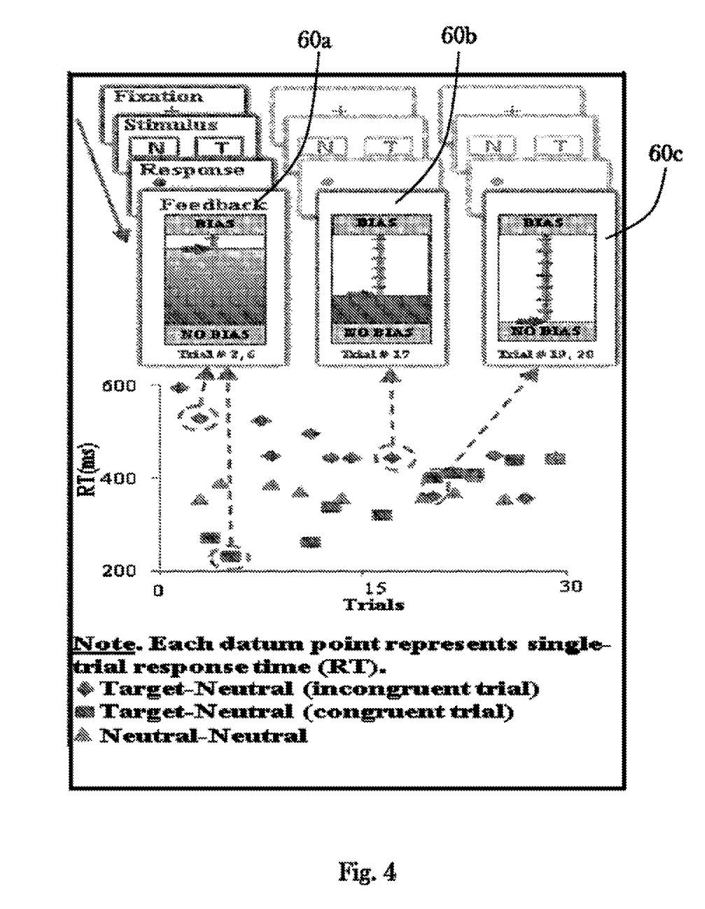

FIG. 4 also shows the steps of the process of FIG. 2 showing how the feedback can be presented through a colored scale showing “bias level” of the subject in real time/near real time. A “high” of “low” attentional bias level of the subject in response to the current task or to all past tasks of the session (e.g. the lower the response time when using a congruent task or the higher the response time when using an incongruent task) will be graphically indicated through the scale. FIG. 4 illustrates three bias levels associated with three different tasks: (i) a first high bias level 60a indicative of a high response time score in the congruent task and low response time score in the incongruent task; (ii) a second bias level 60b lower than the first one 60a due to slightly higher response time score in the congruent task and slightly lower score in the incongruent task in relation to 60a; and (iii) a third bias level 60c representing a low bias level indicative of relatively high response time score in the congruent task and low response time score in the corresponding incongruent task as illustrated in the graph in FIG. 4.

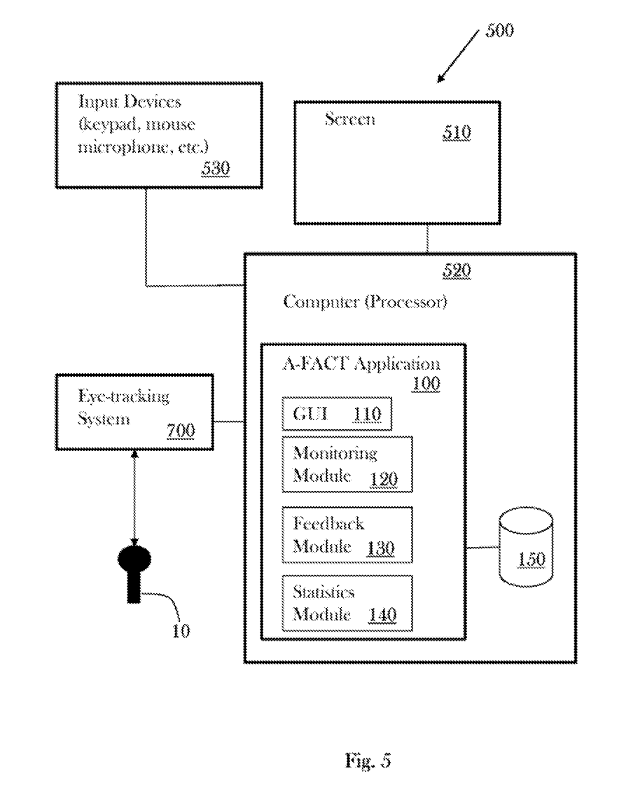

Reference is now made to FIG. 5, which schematically illustrates a system 500 for monitoring biased attention allocation of a subject 10 and provides feedback to the subject 10 indicative of the monitored attention allocation, according to some embodiments of the present invention. As illustrated in FIG. 5, the system 500 includes Attention Feedback Awareness and Control Training (AFACT) application 100, which may include software and hardware components, at least one computer processor such as processor 520 enabling operating the AFACT application 100, receiving input data from various input devices and systems such as through standard computer input devices 530 e.g. a keypad, a microphone, a computer mouse, a touch screen, or any one or more other input device, and outputting devices such as a screen 510, a speaker (not shown) and the like.

Optionally, depending on system 500 definitions and AFACT application 100 capabilities, the system 500 further includes one or more stimuli and/or detection systems such as an eye-tracking system 700, as illustrated in FIG. 5 for either applying the sensory stimulus and/or measuring the subject's 10 attention allocation related response to the applied stimulus.

Any measuring device can be used to measure attention allocation related parameters of the subject in response to a given stimulus. The measuring device can measure, for example, physiological response of the subject to the respective stimulus and output data to the computerized system processor 520 indicative of the measured response to the stimulus for allowing the processor 520 to use this data to deduce the values of respective one or more attention allocation indices therefrom. The indices may be the parameters themselves where each attention allocation index is a parameter type and/or a calculation based on the measured parameter. For example, the measured parameter may be the time it takes the subject to respond to a given task related to a visual stimulus of presented images, where this measure of time is used to calculate the subject's respective bias level value of the respective training session or task, where the bias level is the attention allocation index assessed by the computer processor 520.

In this example an eye-tracking system 700 is used for measuring the subject's physiological response to visual stimuli associated with biased attention allocation. As known in the art, eye-tracking systems use laser technology to track the pupils of the subject 10 and therefore deduce the eyes' focal point, gaze duration and other such parameters as mentioned above. These parameters allow automatic measuring of the response time of the subject 10 to the visual stimulus. For example, when using the dot-probe, spatial cueing task and/or visual search task paradigms all involving using visual stimulus (images presented on-screen, the response to the visual stimulus in relation to the settings and conditions of the specific task can be automatically measured through the eye-tracking system 700 by locating the gaze of the subject 10 at each given moment and enabling to measure/calculate the time it takes the subject 10 to move his gaze from one location to another in response to the task requirements, for example.

For example a commonly used eye-tracking system such as a Tobii TX 300 (300 Hz) binocular system may be used. Consistent with norms, fixation may be operationalized as gaze within a radius of 30 pixels for at least 100-ms, averaged across both eyes, measured separately via corneal reflection. Pupil size responses (dilation) can also be calculated from the eye-tracking system 700 measures to index degree of emotional arousal to target stimulus (threat, smoking) relative to neutral stimulus; average pupil size in the cross-fixation inter-trial intervals will serve as the baseline. This means that the measurements of the eye-tracking system allow deducing or extracting a variety of response types in addition to the response time measurement such as the aforementioned dilation parameter, which can indicate a psychological response to the stimulus.

According to some embodiments of the present invention, the AFACT application 100 may include one or more training programs each training program includes a set of tasks associated with a specific attentional bias type and a predefined training method (e.g. dot-probe/visual search/spatial cueing—based attention allocation monitoring and feedback training program).

According to some embodiments of the present invention, as illustrated in FIG. 5, the AFACT application 100 includes a graphical user interface (GUI) 110, a monitoring module 120, a feedback module 130, and a statistical module 140. The GUI 110 allows presenting data over the screen 510 and receiving input data from the various computer input devices 530 through a predefined GUI 110 platform. For example the GUI 110 includes predefined input fields such as personal details fields—allowing the user to input details such as his/her age, weight, height, hobbies, variables relevant to bias or to the behaviors impacted by bias or attention allocation, and the like. The bias field may be a selection field requiring the subject 10 to select a bias type out of a predefined list (e.g. addiction type, threat type and the like). Each such field may be associated with a different training program allowing the subject 10 to be trained according to his/her special needs and attentional bias problems. The computerized system can also select the bias type(s) and feedback type for subject outcomes based on measured attention allocation (bias) to various cue types prior to delivery of feedback with respect to those or other cues. The GUI 110 may optionally also allow the subject 10 to select the stimulation type (e.g. auditory or visual) and/or the training goal (e.g. reducing biased behavior in response to the selected bias's cues or increasing biased behavior e.g. for improving instinctive responsive behavior or desired attentional allocation or related behaviors). Once the subject 10 or any other user such as a trainer inputs all or some of the fields such as personal details of the subject 10 and selects the bias type and training goal, the AFACT application 100 selects and retrieves a suitable training program associated with at least one of the details/parameters of these fields from one or more training programs databases such as database 150, which is stored in the memory of the computer 520.

According to some embodiments of the invention, the monitoring module 120 enables measuring parameters associated with the attention allocation of the subject 10 during each task of each training session according to the layout of the system and the training program. For example, if using eye-tracking, the monitoring module 120 enables communicating with the eye-tracking system 700 for receiving data related to measured gaze focusing and duration therefrom for calculating and presenting response time and other optional attention allocation related parameters. The monitoring module 120 further enables performing one or more diagnostic exercises to the subject 10 for assessing his/her most dominant attentional biases for allowing each subject to train a bias type that is most suitable for his/her tendencies. The diagnostic exercises may include tests that are based on known paradigms for identifying and measuring the attentional biases of the subject 10 such as the aforementioned dot-probe, visual search as spatial cueing paradigms.

According to some embodiments of the present invention, the feedback module 130 allows providing one or more feedbacks in real time to the subject 10 indicative of the measured attention allocation by, for instance, using visual indication through a bias scale, indicative of the response time of the latest task. the GUI 110 may allow the subject 10 or any other user to select and define the feedback presentation/outputting. For example, the visual presentation of the feedback may be a default while the GUI 110 allows the user/subject to select an additional/alternative channel for outputting the feedback such as through audio feedback. In this way both the visual representation of the feedback (e.g. response time number or response time performances histogram) and an audio output message announcing the response measures as the attention feedback can be provided to increase the impact (e.g. the psychological impact) of the feedback over the subject 10 during training for increasing his/her awareness to his/her attention allocation even further.

The statistical module 140 may enable recording monitored attention allocation indices values of the subject(s), calculating statistical values by using the recorded retrieving statistical data associated with each training program (optionally in respect to the inputted personal details of the subject such as gender, age, nationality, religion and the like) to allow accumulating and optionally presenting statistical information that relates to the subject of attention allocation training. This may allow, for example, to keep records of training performances of each subject 10 (e.g. in relation to each specific bias type trained) and follow his/her awareness development (checking whether the training helped the subject 10 to increase or decrease his attention allocation in relation to the bias type and to the selected training goal). In this way the training application 100 may allow supporting a process of training for each subject 10 including follow up abilities that can support both researching the effects of feedback and the various training programs and paradigms on attention allocation as well as help each subject 10 or a caretaker thereof to follow the subject's progress and/or effectiveness of each training program over time.

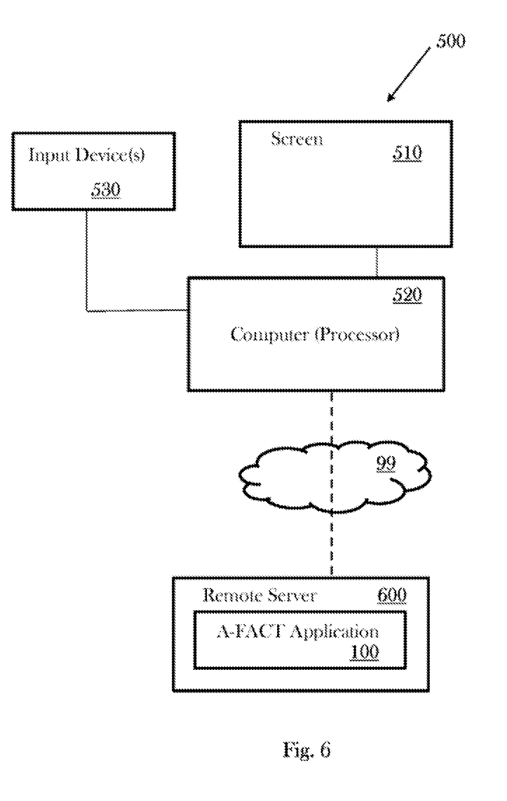

According to some embodiments of the present invention, as illustrated in FIG. 6, the AFACT application 100 may be operated through a remote server 600 communicating with client computers such as computer 520 through one or more communication links such as through an internet link 99 enabling thereby a multiplicity of end user to operate the AFACT application. This means that the AFACT application allows supporting operation of multiple training programs used by multiple subjects/user through their end computers or other electronic devices (e.g., smartphone, tablet device). In this case the application 100 may support an interactive website enabling subjects (users) to link thereto for training awareness to biased attention allocation, while the website allows recording training sessions' scores and thereby perform various statistical evaluations and calculations.

The methods and systems of the present invention are expected to increase awareness to biases and afford the capacity to self-monitor attentional allocation through learning a new association(s) enabled, uniquely, by real-time feedback, through AFACT training Initially, an individual receives feedback about her/his (biased) allocation of attention and her/his typically overlearned, unintentional, or/and unmonitored allocation of attention. A person thus learns a new association between (i) the events immediately preceding (e.g., exogenous or endogenous context or bias-facilitating cues such as thoughts, physical sensations, environmental context), the visual object(s) and other sensory stimuli to which attention is allocated, and immediately preceding biased/preferential allocation (e.g., thought, emotion, and/or physical cue) and (ii) the degree to which her/his attentional allocation was biased (bias level). This new learning occurs in real-time over the course of repeated trials via associative and operant conditioning. Elevated awareness of biased attentional allocation is expected to lead to increased capacity of the subject to regulate or control attentional allocation and thereby reduce bias. Contemporary thinking regarding the primary mechanism underlying attentional bias, such as the inability to disengage attention (e.g., from threat cues), is driven by dysregulation in attentional control. Thus, the methods and systems of the present invention provide a novel means to train attention that will likely affect biased allocation by targeting the core mechanisms underlying bias.

Bias Awareness and Moderation of the Psychopathogenic Effects of Attentional Bias: In addition to facilitating increased control of attentional allocation, elevated awareness of bias is furthermore expected to moderate (buffer) the psychopathogenic effect(s) of attentional bias (e.g., drug-seeking in addiction maintenance, escape-avoidance in anxiety psychopathology). Thus, the proposed salutary moderation or buffering effect of attentional allocation awareness training as described above on potentially uncontrollable components of biased attentional allocation (e.g., threat detection underlying facilitated attention) is also expected to operate through an additional mechanism. Specifically, bias awareness and self-monitoring is expected to enable the capacity to engage in alternative behaviors to those typically driven by unmonitored attentional bias (e.g., behavioral choice, top-down behavioral inhibition in contrast to typically unmonitored, automatic pursuit of negative reinforcement opportunity), and thereby neutralize the typically automated psychopathogenic effects of attentional bias. Moreover, this additional salutary mechanism of feedback-facilitated awareness may, for some, serve as a “second-line of defense” in the event of transient attentional dyscontrol. Finally, through increased awareness and capacity to self-monitor bias and to regulate and control attentional allocation, attention allocation awareness training is further expected to reduce the development and maintenance of multiple prevalent forms of psychopathology and addictions and therefore potentially serve as a prevention tool for preventing subjects from developing or enhancing psychopathology and/or addictions.

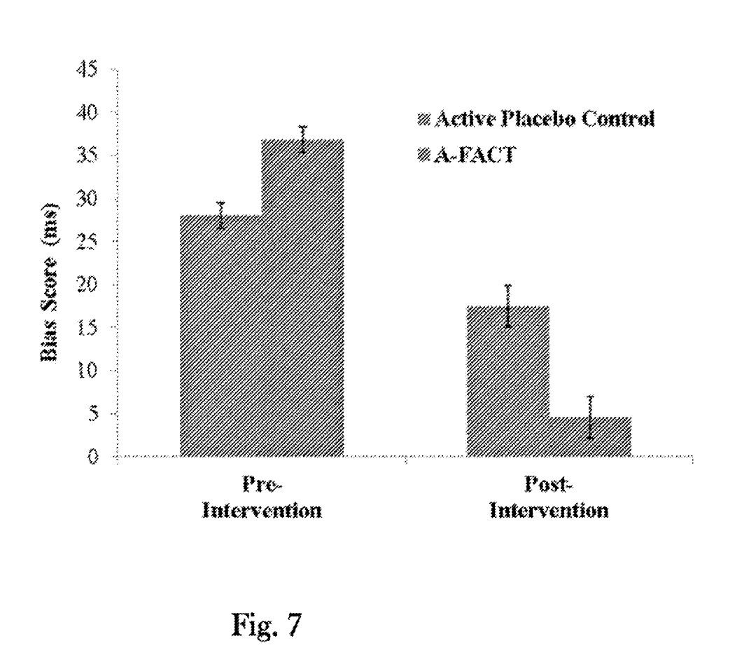

Reference is now made to FIG. 7, which shows experimental results of AFACT using the dot-probe paradigm for monitoring the attention allocation of a subject, providing real time feedback to the subject after each task in each training session. In a randomized control experimental design, we tested AFACT relative to an active placebo control condition among 40 anxious adult subjects demonstrating attentional bias to threat stimuli. The active placebo control condition was identical to the AFACT condition except that in the former no real-time feedback was delivered to the subjects. Accordingly, in so far as AFACT engenders awareness-of-attention and thereby self-regulatory control, then bias reduction should result. Indeed, results shown in FIG. 7, demonstrated randomized control experimental evidence that AFACT ameliorates attentional bias.

Additional or alternative methods and paradigms can be used to monitor attention allocation and the four main paradigms mentioned above are but a few examples to monitoring attention allocation via an exemplary type of visual stimulus. As mentioned above, other types of visual stimuli based paradigms can be used and/or other types of sensory stimuli such as auditory and/or tactile stimuli and other types of measuring the response to the external or internal stimuli (e.g., the stimuli as a physical event) may be used such as brainwave based measuring techniques and devices.

Additionally or alternatively various types and techniques may be used to provide the feedback such as visual, auditory and the like. A combination of more than one type of feedback can be used to increase effectiveness thereof.

Various technologies may be used to provide both monitoring and feedback such as biofeedback and/or neurofeedback based technologies utilizing these technologies to provide feedback on attention allocation in relation to specific one or more attention biases.

For example in additional or alternative embodiments of the present invention, one or more devices that measure physical responses to the applied stimulus may be used without requiring the subject to actually perform a task to measure his/her allocation attention to the respective stimulus. For example a magnetic resonance imaging (MRI) device, electrocardiography (ECG) device, etc. may be used to measure physiological processes occurring in the subject's body (such as the subject's brain or heart respectively), in real time when applying the stimulus (e.g., visual, tactile, auditory, olfactory and/or gustatory (taste) stimulus). The feedback is given to the subject in real time or near real time immediately after the stimulus is applied and the subject's physical reaction to the stimulus is measured and/or calculated.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments and/or by the following claims. For example, notwithstanding the fact that the elements of a claim are set forth below in a certain combination, it must be expressly understood that the invention includes other combinations of fewer, more or different elements, which are disclosed in above even when not initially claimed in such combinations. The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

Although the invention has been described in detail, nevertheless changes and modifications, which do not depart from the teachings of the present invention, will be evident to those skilled in the art. Such changes and modifications are deemed to come within the purview of the present invention and the appended claims.

1. A computerized method for monitoring and training attention allocation, said method comprising:
a) applying at least one sensory stimulus over a human subject, using at least one stimulation device, said sensory stimulus is associated with at least one attentional bias;
b) measuring at least one attention allocation index of the subject by measuring response of the subject to said applied sensory stimulation, using at least one measuring device that measures physiological response of the subject to the respective stimulus, wherein data indicative of the measure response to the stimulus is outputted by said respective measuring device;
c) deducing at least one attention allocation index measure from the measured physiological response to the stimulus, using at least one computer processor; and
d) outputting an attentional feedback indicative of the deduced at least one attention allocation index of the subject, said feedback is outputted in real time or near real time, using at least one output device.

2. The method according to claim 1, wherein said sensory stimulus comprises at least one of: visual stimulation, auditory stimulation, tactile stimulation, olfactory stimulation, and/or gustatory stimulation.

3. The method according to claim 1, wherein said feedback is a visual feedback, auditory feedback, and/or tactile feedback.

4. The method according to claim 1, wherein said attention allocation index comprises at least one of:
a) reaction time indicative of the time it took the subject to respond to the applied stimulus;
b) task time, indicative of the time it took the subject to fulfill a requested task associated with the respective applied stimulus; and/or
c) at least one physiological measure indicative of physical response of the subject to the stimulus.

5. The method according to claim 1, wherein said training is carried out by using a training program that comprises a set of training sessions each session includes a set of tasks comprising:
a) providing the subject with at least one sensorial stimulation;
b) measuring at least one attention allocation index by measuring the subject's response to said stimulation; and
c) outputting a feedback indicative of said measured at least one attention allocation index.

6. The method according to claim 1, wherein said attention allocation index measuring is carried out by using at least one predefined attention allocation scheme; said scheme is based on at least one of: a dot-probe paradigm; a spatial cueing paradigm; a visual search paradigm; and/or modified stroop task; interference based schemes, attentional inhibition based schemes.

7. The method according to claim 1, wherein said measuring is carried out by using an eye tracking system that measures the subject responses to visual stimuli.

8. The method according to claim 1, wherein said measuring device comprises one of: an eye-tracking system, a magnetic resonance imaging (MRI) device, a psychophysiological device, an electroencephalography (EEG) or an event-related potential (ERP) device.

9. The method according to claim 1, wherein data from said measuring device, indicative of at least one measure detected thereby, is transmitted to at least one computer including at least one processor for identifying attention allocation indices values, said computer further allows outputting a feedback indicative of said measures by transmitting signals to at least one output device.

10. The method according to claim 1, wherein said outputting of said attention allocation feedback comprises presenting at least one graphical representation indicative of a bias level of the subject associated with a respective attention allocation index measure.

11. The method according to claim 10, wherein said graphical representation includes at least one of:
a) a graphical indication representing statistics of the bias level or related nature of the bias of the subject and/or of multiple subjects in relation to all previously measured responses; and/or
b) a graphical indication of the attention allocation of the subject in relation to the last stimulus.

12. The method according to claim 1 further comprising recording values of measured attention allocation indices and calculating statistical values associated therewith.

13. A system for monitoring and training attention allocation, said system comprising:
a) at least one stimulation device capable of applying at least one sensory stimuli;
b) at least one computer processor enabling to operate a designated Attention Feedback Awareness and Control Training (AFACT) application, said AFACT application enables monitoring at least one attention allocation index of a human subject by measuring response of the subject to applied sensory stimuli, said sensory stimuli is associated with at least one attentional bias, and outputting an attention feedback indicative of said measured at least one attention allocation index of the subject in real time or near real time; and
c) at least one output device for allowing outputting said feedback.

14. The system according to claim 13, wherein said AFACT application applies stimulus through at least one output device connected thereto and performs said monitoring by measuring or receiving measured response time to said stimulus.

15. The system according to claim 13 further comprising at least one measuring device that measures physiological response of the subject to the respective stimulus, wherein data indicative of the measured response to the stimulus is outputted by said respective measuring device and is used to deduce the respective at least one attention allocation index therefrom.

16. The system according to claim 15 wherein said measuring device comprises at least one of: and eye-tracking system, a magnetic resonance imaging (MRI) device, a psychophysiological device, or an electroencephalography (EEG) and/or event-related potential (ERP) device.

17. The system according to claim 13 further comprising a database storage comprising a multiplicity of training programs each adapted to a different attentional bias type.

18. The system according to claim 17, wherein each said program is designed according to at least one additional input parameter inputted through predefined input fields provided by said AFACT application via a designated graphical user interface, wherein once these parameters are inputted, the AFACT application automatically selects and executes a suitable training program from said database according to said input parameters.

19. (canceled)

20. The system according to claim 13 further comprising at least one input device for allowing the subject to respond to said stimulation therethrough, said input device includes at least one of: a touch screen, a computer mouse, a keypad, wherein said processor allows measuring said at least one attention allocation index by measuring response time of the subject.

21. (canceled)

22. (canceled)

23. A system for monitoring and training attention allocation of a subject, said system comprising:
a) at least one stimulation device capable of applying at least one sensory stimuli, said stimulation device comprising at least one of: a computer screen, an audio speaker;
b) at least one computer processor enabling to operate a designated Attention Feedback Awareness and Control Training (AFACT) application, said AFACT application enables monitoring at least one attention allocation index of a human subject by measuring response of the subject to applied sensory stimuli, said sensory stimuli is associated with at least one attentional bias, and outputting an attention feedback indicative of said measured at least one attention allocation index of the subject in real time or near real time; and
c) at least one output device for allowing outputting said feedback, said output device comprises at least one of: a computer screen and/or an audio speaker.
